(19) United States
(12) Patent Application Publication
(10) Pub. No.: US 2022/0174943 A1
(43) Pub. Date: Jun. 9, 2022

(54) ORGAN AND FLUID PRESERVATION AND TRANSPORTATION CONTAINER AND DOCKING SYSTEM

(71) Applicant: Desert Valley Tech Inc., Mesa, AZ (US)

(72) Inventors: Burt Skiba, Mesa, AZ (US); Travis Witzke, Scottsdale, AZ (US); Clint Solomon, Gilbert, AZ (US); Jeffry Skiba, Chandler, AZ (US)

(21) Appl. No.: 17/113,469

(22) Filed: Dec. 7, 2020

(51) Int. Cl. A01N 1/02; F25B 21/04; A61L 2/10; A61L 2/26

(52) U.S. Cl. CPC A01N 1/0273; F25B 21/04; A61L 2202/23; A61L 2/26; A61L 2202/11; A61L 2/10

(57) ABSTRACT

The present application provides a unique way for storing biomaterials, blood, blood products, vaccines, organs in an organ and fluid preservation and transportation system. The organ and fluid preservation and transportation system can comprise a docking system, a container system comprising an upper container, an inner container, an outer container, electronics, and a heating/cooling module. The upper container can attach to the outer container, and/or inner container and create an airtight seal with the environmental conditions staying consistent when sealed wherein the container system can be docked to the docking system wherein the docking system heats or cools down the container system.

### CROSS REFERENCE TO RELATED APPLICATIONS

The present application claims the benefit under 35 U.S.C. 119 of U.S. Provisional Patent Application Ser. No. 62/944,475 filed Dec. 6, 2019. The U.S. Provisional Patent Application Ser. No. 62/944,475 is hereby incorporated by reference in its entirety.

### TECHNICAL FIELD

The present invention relates to a portable environmental storage, preservation, and a transportation container usable by individuals that have a need to transport, store and monitor biomaterials at a set environmental condition. More particularly, a portable, actively cooled or heated storage and transportation container that can be tracked and connected to a computing device allowing a user to dock the device and monitor, set and control the environmental condition in the device's compartment and send an alert should the contents go out of allowable range or set point.

### BACKGROUND

Various types of devices are used for safely storing and carrying vaccines, blood, plasma, biofilms, and organs collectively referred to as biomaterials or biologics. For example, military field medics carry medical equipment that can weigh from 50 lbs. to 80 lbs. including bandages, saline, tourniquets, tubing, gauze, and other lifesaving materials. However, current devices require the field medic to carry multiple devices and to have access to a freezer or refrigerator to keep lifesaving biomaterials at the recommended storage conditions. Because each of these biomaterials have a recommended storage condition to be viable, multiple devices are needed to accommodate each storage condition. For example, human organs and vaccines must be transported at different conditions to keep the vaccines potent, and human organs from dying than blood products, and pharmaceuticals. Current mobile storage devices for lifesaving biomaterials are passively cooled meaning the device use ice, or ice products such as ice packs with thick Styrofoam lined shells. The passive storage of biomaterials has a number of disadvantages including, ice melting causing condensation on the organ or blood product spoiling the product. Ice will additionally create inconsistent temperature variation within the container causing an uncertainty of whether the biomaterials are viable or as potent. This device will enable tracking of the contents real time delivering informatics on the condition and location so the viability of the biologics will be known up to the time of use.

Blood products such as plasma, platelets, red blood cells, or whole blood usually come in blood bags and must be stored at specific temperatures to keep fresh and usable. Using ice, or ice-like packs can cause inconsistencies within the temperature range of the blood bag creating uncertainties of whether the blood is safe for transfusion. The blood product usually delivered in a blood bag must be in contact with the ice or ice pack to stay cool, and the areas not in contact with the blood bag, or the ice becomes separated from the blood bag the blood or plasma product could spoil. In addition, the storage environment of different type of blood products varies in temperature, for example whole blood must be stored between 1 to 6 degrees Celsius, plasma must be stored at −18 degrees Celsius or colder, and platelets must be stored between 20 to 24 degrees Celsius. Using ice or ice packets can only give off a baseline temperature of around 0 degrees Celsius. There is not currently a product on the market today that can adjust to a set temperature and to different required environmental conditions to fit the different types of blood products, organs, and vaccines in remote locations.

A further disadvantage of the use of ice or ice packets for storing biomaterials is that ice melts. This melting limits the available storage space and time, causing condensation, causing the need to dispose of the water or water packets, the user must now find replacement ice during transportation to keep items cool. There must be an ample supply of ice as the blood products, organs, and vaccines are transported from location to location. Furthermore, ice is generally not sterile, and if the blood products, organs, or vaccines are surrounded by ice, contamination during transportation may arise making the products unusable. Microbes may form and cover the container, and the surrounding walls transporting those microbes to the blood products, organs, or vaccines when removed and put into the patient.

Therefore, it would be advantageous to provide a cold or hot chain storage container that can track and send alerts and can actively control a cooling element that keep items at a set temperature, humidity level and a controlled environment.

### SUMMARY

Aspects disclosed herein relates to an organ and fluid preservation and transportation container and docking system. An organ and fluid preservation and transportation container can comprise an outer shell, an inner container, an insulative layer, a cooling element and a lid. The outer shell can surround or encapsulate the inner container wherein the inner container can be an insulative material. The lid can further comprise an access port, a computing system, communications port for the computing system, environmental sensors, dispersion plates, an insulative layer, and in certain embodiments a cooling element.

A docking system can comprise a holding element, a cooling element, and computing system wherein the holding element can removably attach to the docking system the organ and fluid preservation and transportation system. When docked the organ and fluid preservation and transportation container can lift or lower it onto a heating/cooling module such as a thermal electric cooler or liquid cooling system. The computing system can transmit data from the container to the cloud allowing the user to see in real time on a mobile computing device the environmental conditions that the container has seen and is currently seeing. The heating/cooling module can be attached to a distribution plate and can further comprise a heat sink, and a fan.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 shows an isometric view of an organ and fluid preservation and transportation system in accordance to one, or more embodiments;

FIG. 2 shows a closed isometric view of an organ and fluid preservation and transportation system in accordance to one, or more embodiments;

FIG. 3 shows a side section view of an organ and fluid preservation and transportation system in accordance to one, or more embodiments;

FIG. 4 shows another embodiment as an exploded isometric view of an organ and fluid preservation and transportation system in accordance to one, or more embodiments;

FIG. 5 shows a side view of an organ and fluid preservation and transportation system connected to a drone in accordance to one, or more embodiments; and

FIG. 6 shows a front view of an organ and fluid preservation and transportation system connected to a drone in accordance to one, or more embodiments;

FIG. 7 shows a front view of an organ and fluid preservation and transportation system connected to a drone in accordance to one, or more embodiments;

FIG. 8 shows a front view of an organ and fluid preservation and transportation system connected to a drone in accordance to one, or more embodiments;

FIG. 9 shows a front view of an organ and fluid preservation and transportation system connected to a drone in accordance to one, or more embodiments;

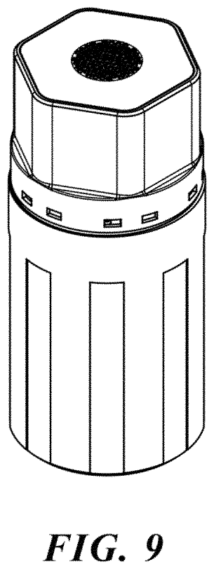

FIG. 10 shows another embodiment front view of an organ and fluid preservation and transportation system connected to a drone in accordance to one, or more embodiments; and

FIG. 11 shows cross sectional view of FIG. 10 of an organ and fluid preservation and transportation system connected in accordance to one, or more embodiments.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

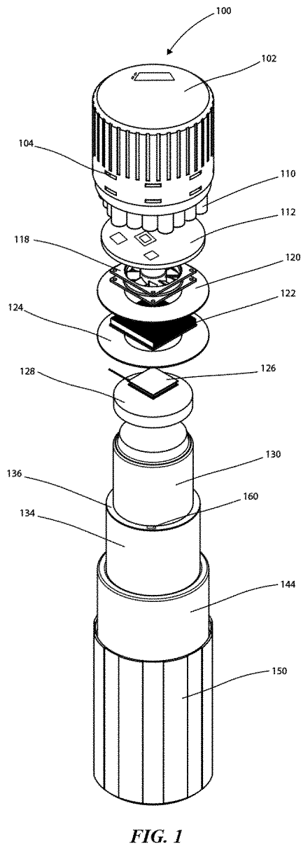

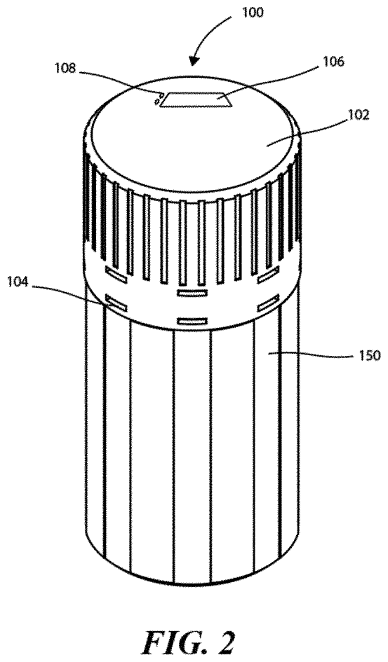

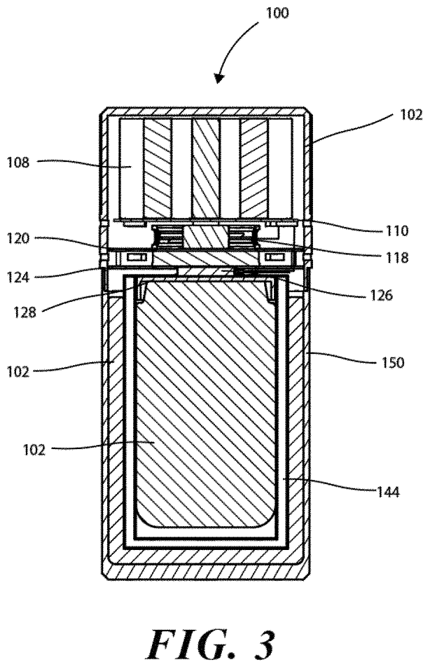

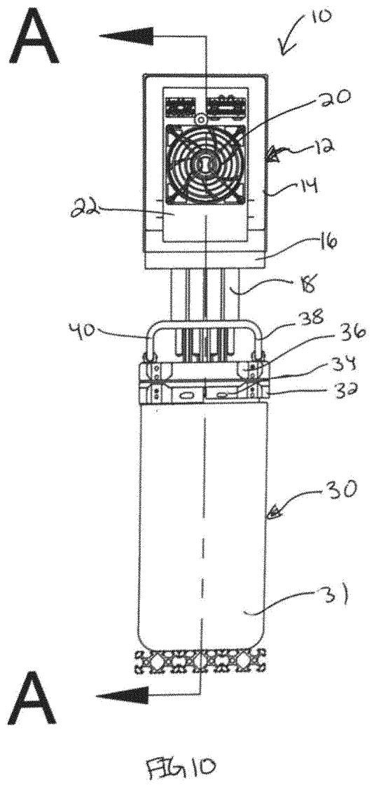

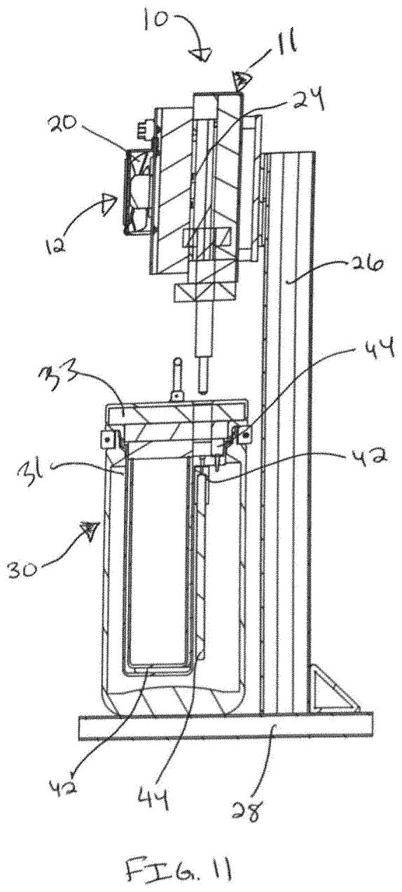

Referring initially to FIG. 1 through 3, an exploded isometric view, front isometric view, and cross-sectional view of an organ and fluid preservation and transportation system is shown generally at 100 showing the docking station in FIG. 10 and FIG. 11. An organ and fluid preservation and transportation system 100 can comprise container system 101 comprising an upper container 102, a lower container 150, an insulative layer 144, an inner container 134 and a docking system 200.

The docking system 200 can comprise a heating/cooling system 115 which comprises a fan 118, one or more heat sink 122, a heating/cooling module 126, and a distribution plate 128. In the preferred embodiment the heating/cooling system is in the docking station, but in other the docking station 200 can further comprise electrical system 112 wherein the electrical system can comprise, but not limited to, a wireless communications device, a global positioning system (“GPS”) module, a microcontroller unit (“MCU”) 246, a charging controller, one or more switches, one or more general purpose input/output (“GPIO”), a memory medium, and a printed circuit board (“PCB”). The PCB can be manufactured out of a single sided, double sided, multilayered, flex, or the like. The PCB can have a system on a chip (“SOC”) 248 having a microcontroller unit (“MCU”) 246, and a communications module 244 wherein the MCU can execute programmed instructions and can have embedded software that can be stored and/or pull from a memory medium (not shown) which can be integrated on either on MCU or on the PCB. The memory medium (not shown) can be integrated within the PCB 122, and/or can be its own independent system, wherein the memory medium can store data, store algorithms to control the different systems within the organ and fluid preservation and transportation system 100.

The electrical system 112 can be attached to the docking system 200 or can be integrated within the docking system. The wireless communication device (not shown) can be such as, for example, BLUETOOTH Low Energy (BLE), a BLUETOOTH module, Wi-Fi module, IEEE 802.15.4, Z-Wave, Single/Dual Mode Radio Chip. The wireless communication device (not shown) can transmit data packets to a cloud system, a smartphone device, or a static computing device such as a personal computer, military command/headquarters computing device, tablet, or the like of the status of the organ and fluid preservation and transportation system.

The GPS module can use a global navigation satellite system to locate, provide geolocation and time information about the organ and fluid preservation and transportation system as it is being moved from location to location creating a log of information stored in a memory medium, or transmitted to a remote computing device. The GPS module can transmit the information to a user wherein the user can in real-time track the system, the system's contents, and the environmental conditions. The GPS module can be integrated circuit chip, NMEA protocol, TSIP, TAIP, Skytraq binary, or the like. The GPS module can relay information such as location or coordinates to a computing device that is located nearby, or a base that is located somewhere around the world, allowing the user to send their location when a soldier is injured, or when the vaccine is used, or to a hospital so it can track the location of the organ. In certain embodiments, the GPS location can be external such as in a drone or UAV system wherein the drone GPS can send the device's current location.

The MCU 246 can control the entire organ and fluid preservation and transportation system such as, for example the GPS memory module, the temperature module, humidity module, battery regulation, fan, power, and all other systems ran by the electronics, or each electronic module system can control itself. The MCU can be for example, an 8-bit MCU, 16-bit MCU, and 32-bit MCU wherein the MCU can be embedded memory or external memory. The MCU can have embedded algorithms that monitor, diagnose and control the charging controller, at least one sensor 160, at least one switch, a fan 118, and the heating/cooling module 126.

The heating/cooling module 126 can be attached to a distribution plate 128 by thermal insulation material or a plurality of fasteners, and can further comprise a heat sink 122, and a fan 118. In certain embodiments, the distribution plate 128 can be omitted and the heating/cooling module can connect to or attach to, or touch the inner container 134 to separate the hot/cold air and draw the heat or cold air away from the heating/cooling module 126 a top divider plate 120, and a lower divider plate 120 separate the heatsink 122 and the fan 118 allowing the air to flow past the heat sink and can be drawn away from the heat sink to above the top divider plate forcing the hot or cold air out of the upper container 102. The upper container 102 can have a plurality of holes 104 around the circumference of the upper container wherein the holes can be located above the top divider plate 120, and below the top divider plate, or the holes can be placed on the lower container or can be omitted. The plurality of holes 104 can have an optional filter (not shown) that can keep dust and particulates out of the upper container 102 keeping the electronics and other components clean.

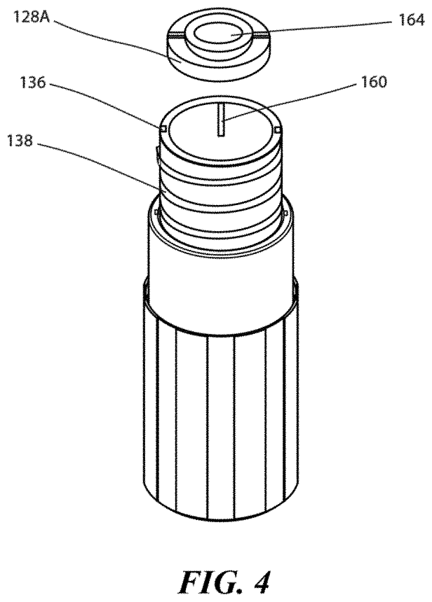

The fan 118 can have a thermal insulative material layer between its bottom surface and the heating/cooling module 126 top surface allowing the heat or cool to be drawn away from the heating/cooling module. The fan 118 can be circular, radial, forward inclined blades, or the like wherein the fan can be ran in both a forward or reverse direction pulling air away, or pushing air towards the heating/cooling module 126. In certain embodiments, the fan 118 can be omitted from the system, or the fan can be replaced with a liquid cooling system that can comprise antifreeze or water in tubing, a pump, and a heat sink, and in other embodiments a fan can be included with the liquid cooling system. In other embodiments, as shown in FIG. 4, a tubing 138 can be wrapped around the outer walls or on the inner walls of the inner container 134 wherein the tubing can carry the refrigerant or water from a pump 164 that can cycle or flow the refrigerant or water past the cooling/heating module 126 and through the tubing causing the environmental temperature to decrease or increase the inside or walls of the inner container.

The distribution plate 128 can be connected to the upper container 102, or can be connected through the upper divider 120 and the lower divider 124, or it can be connected to the upper divider or lower divider. The distribution plate 128 can be in the shape of a circular, square, rectangle, hexagon, or the like, and it can be in contact with the inner container 134 wherein the distribution plate can transfer the thermal cooling, or thermal heating from the cooling/heating module 126 to the inner container spreading the cold or heat evenly to the inner container. The distribution plate 128 can be manufactured of stainless steel, aluminum, brass, bronze, or any material that can act as a heat transfer material. In certain embodiments, the distribution plate 128 can be omitted, or the distribution plate can have heat transfer material such as vinyl, kapton tape, aluminum paste or the like. In other embodiments, the distribution plate 128 can act a seal between the upper container 102 and the inner container 134 wherein it can create an airtight seal within the inner container. The distribution plate 128 can be omitted in certain embodiments and the heating/cooling module 126 can be in direct contact with the inner container 134.

The heating/cooling module 126 can be a Peltier module, evaporative cooler, liquid cooling or other cooling or heating module which can be for example, high performance, high or low temperature, micro cooler, multi-stage, rounded, thermoelectric cooler, cold plate coolers, liquid coolers, or the like. The heating/cooling module 126 can be controlled by the electronics 112 wherein the user can set a desired temperature from about or lower than −40 degrees C. to about or high than 80 degrees C. The user can set the temperature remotely through a mobile computing device, or on the organ and fluid preservation and transportation system itself such as a through an LED screen 106 located on the upper container 102, or on the lower container 150, or through push buttons. The temperature can be monitored and tracked, and data points collected on a mobile computing device, on the MCU and memory medium, or a remote computing device. At least one temperature sensor (not shown) can be placed throughout the inner container 134, and/or lower container 150 wherein an array of temperatures throughout the system can be established to measure the temperature of the contents that are placed inside the inner container. The temperature can be tracked and then sent to a user through WiFi or other wireless communications technology. A temperature sensor can be placed on the upper container 102, and/or lower container 150 wherein the outside ambient temperature can be monitored and then compared to the temperature on the inside of the inner container 134. The temperature sensors can be placed in or on the inner surface of the outside container measuring the temperature difference between the outside temperature, the first insulation layer, and the inner container. In certain embodiments, a temperature sensor can be placed between the inner and outer walls of the inner container 134, and in other embodiments the temperature sensor can be omitted.

The switch (not shown) can turn on and off the heating/cooling module 126, the fan, and can monitor when power is needed to the PCB and MCU wherein the switch can be for example, control switches, magnetic switch, multi-directional switch, programmable switch, or the like. By having the heating/cooling module switch 126 on and off when a temperature drop is needed the batteries can be depleted only when power is needed to the whole system. In some embodiments, the switch can be omitted or can be replaced with algorithms using the MCU and can triggers an event and turn on or off the heating/cooling module 126. The one or more general purpose input/output (“GPIO”) can be on the PCB or within the MCU wherein the GPIO can be integrated circuit, and/or board level GPIOS. The GPIOs can be connected to one or more sensors, the fan 118, the heating/cooling module 126, or the like within the organ and fluid preservation and transportation system 100. The data collected can be uploaded to a cloud system and analyzed for cargo viability and allow physicians or the user to see in real-time at remote locations what conditions the cargo is in.

The electronics 112 can further be comprised of a light sensor (not shown) which can be attached through wires to the MCU or GPIOs, and can be attached to the distribution plate 128, or the lower divider 124, or anywhere within the organ and fluid preservation and transportation system 100 where ambient light can enter into the system. The light sensor (not shown) can recognize when the upper container 102 is off and send a signal to the MCU wherein the detected light can alert a computing system remotely that the organ and fluid preservation and transportation system has been opened. The alert can be given to the user, headquarters, command center, air lift medical helicopter team, or the like. The light sensor (not shown) can trigger a time stamp within the organ and fluid preservation and transportation system 100 when it was opened and when it was closed allowing the user to track the cargo within the inner container 134.

The inner container 134 can be at least one wall or in a preferred embodiment be two walls, an inner wall and an outer wall both having a bottom with a top sealing the inner wall to the outer wall wherein a vacuum can be pulled within the air gap between the inner wall and the outer wall leaving an insulative layer. The inside wall with bottom can be placed into the outside wall with a bottom, and sealed together at the top with a top piece forming a gap between the inner and outer wall, which the air can be evacuated, or different types of gases can be injected into to form an insulative layer. The gases can be for example, Sulfur Hexafluoride, argon, nitrogen, or the like. In other embodiments, the air gap is not vacuumed when being manufactured. The inner container 134 can be an insulative layer to protect and keep the contents that can be stored in the chamber inside the inner container and the inner container can be made of plastics or metals for example, glass filled nylon, Polyethylene Terephthalate, High-Density Polyethylene, stainless steel, aluminum, Inconel, titanium, carbon steel, or the like. The inner container 134 can be placed into and/or supported by an insulative layer 144, or in some embodiments the insulative layer 144 can be omitted and the inner container can be placed into the outer container. In some embodiments, the inner container 134 can be omitted. In embodiments, an inner container 134 can have an outer diameter and/or circumference if not a circle, of at least between 1 inch to 48 inches, more preferably between 2 inches to 24 inches and preferred is 3 inches to 18 inches. The inner container 134 can be for example, circular, square, rectangular, hexagonal, or the like in shape. The insulative layer 144 can be for example, foam, fiberglass, mineral wool, fiberglass, rubber, fiber, thermoplastics, closed-cell foam or the like.

The lower container 150 can surround the inner container and the insulative layer 144, and can be removably attached to the upper container 102. The lower container 150 can have inner diameter and an outer diameter with a bottom. The lower container 150 can have threads on the upper most portion allowing the upper container 102 to be attached to the lower container, or it can be a quick twist to lock into place, or it can have a latching mechanism to latch the lower container to the upper container. In certain embodiments, the upper container 102 can be locked onto the lower container 150 wherein a lock can secure the two together and can keep the two pieces securely locked together during transport. The container as a whole can have a manual pump which can evacuate the air out of the compartment. The lower container 150 can be manufactured from both metals or plastics such as, for example, stainless steel, aluminum, Inconel, glass filled nylon, Polyethylene terephthalate, polycarbonate, High-Density Polyethylene, Polypropylene, or the like. The lower container 150 can have an outer diameter and/or circumference of at least between 1 inch to 48 inches, more preferably between 2 inches to 24 inches and preferred is 3 inches to 18 inches.

In certain embodiments organ and fluid preservation and transportation system 100 can monitor humidity, temperature, pressure, shock, location, time, light, and contaminants within the blood, organ, or vaccine. The electronics 112 can include a pressure sensor, light sensor, temperature sensor, infrared temperature sensor, accelerometer, scanner, and each associated sensor can be placed within the inner container, outer container, and/or upper container. The organ and fluid preservation and transportation system 100 can measure both the ambient temperature, and the temperature within each container level. The electronics can have a global positioning system, Bluetooth Low Energy module, radio-frequency identification or a IR scanner, barcode scanner, QR-code scanner, or the like for scanning blood bags, vaccines, organ bags, and the system can log when the item was placed within the container, log the environmental conditions within and outside of the container, and then report the information to a user's computer system or handheld computing device via Bluetooth, Wi-Fi, or other remote communication system. Scanning the contents can time stamp the contents going into the container, and then upon exiting the system the contents can be scanned again, allowing the system to time stamp the contents with environmental conditions and whether the contents have exceeded their allowable conditions. In addition, the organ and fluid preservation and transportation system 100 can transmit wirelessly or hard wired to a military base, back to a truck with a computing device, or a remote or portable computing device the conditions of the contents and can send an alert if the contents exceed the allowable conditions.

The organ and fluid preservation and transportation system 100 can further comprise a docking station 200 wherein the docking station can accept the organ and fluid preservation and transportation system and keep power to the system while heating or cooling the container. The docking system (not shown) can be attached to such as, for example, a military Humvee or other military truck, ambulance, helicopter, drone, or the like. The docking station can hold the organ and fluid preservation and transportation system in place while allowing the system to transmit to a user the environmental conditions within the system, the status of contents, and the surrounding environmental conditions. The docking system can be attached to the vehicle wherein the vehicle can provide power to the organ and fluid preservation and transportation system. When undocked from the docking system the organ and fluid preservation and transportation system can standalone on its own power source wherein the batteries 110 can keep the heating/cooling module activated and the electrical system processing keeping the contents cool. The docking system can be removed from, or it can be permanently attached the transportation vehicle.

Referring to FIG. 4 another embodiment of an organ and fluid preservation and transportation system shown generally at 100B. The heating/cooling module 126 can have tubing 138 wrapped around the inner container stainless steel with inlet and outlet with coolant. The tubing 138 can be wrap around or can be integrated within the inner container 134 from the top to the bottom of the inner container, and in some embodiment(s) the tubing wrap can go a quarter of the way, halfway, or three-quarters of the way down or up the inner container. The tubing can be attached or connected to a pump wherein the pump 128A can be connected to or attached to the heating/cooling module 126, or the top or the bottom of the inner container, or the outer container, or the upper container and the pump can cycle fluid around the inner container keeping the contents cool. The pump 128A can be for example, coolant pump, water pump, impulse pump, velocity pump, valveless pump, or the like. In certain embodiments, a heat sink can be attached to a pump or placed next to, and a fan can circulate the heat away from the heating/cooling module and/or the heat sink, and/or the tubing. The pump can move refrigerant through the tubing 138 and can be chilled by the heating/cooling module 126, or by a refrigeration unit. The pump can move the refrigerant past the heating/cooling module 126 and keep the inner container 134 at a user's set temperature. The heating/cooling module 126 can keep the refrigerant or tubing at a set temperature, and in certain embodiment(s) can be detachable from the inner container wherein the tubing can have a connection point for the upper half of the heating/cooling module 126 system can be connected to the tubing and the inner casing.

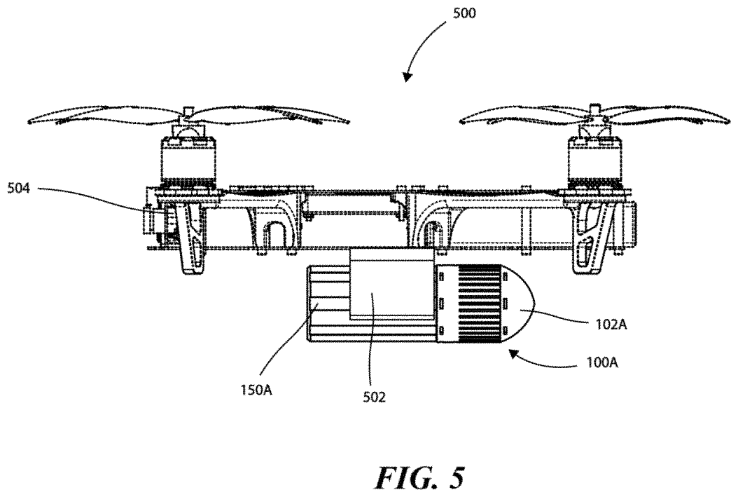

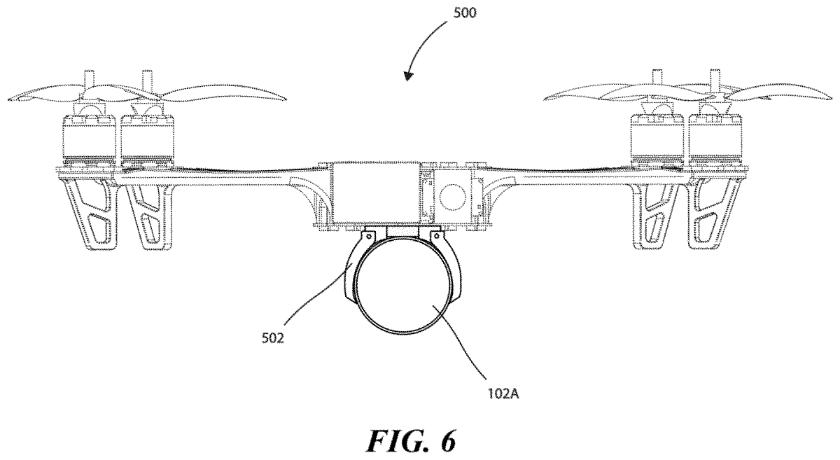

Referring to FIGS. 5-6, another embodiment using a drone or UAV organ and fluid preservation and transportation delivering system 500. In embodiments, the organ and fluid preservation and transportation system 100A can be attached to the drone 504 wherein the attachment 502 can clamp onto, around, or be connected to the outer container 150A. The outer container 150A can have attachment points for the drone attachment 502 point. The drone can draw extra power from the organ and fluid preservation and transportation system 100A power supply, or the organ and fluid preservation and transportation system can draw power from the drone until it is unattached. The organ and fluid preservation and transportation system 100A can be aerodynamic in shape and can use air flow over the outer shell to keep the contents or heating/cooling module 126 at its set temperature wherein the air flow can draw the heat away from the heating/cooling module or it can act as a cooler for the contents within the inner container.

The drone or UAV mechanical device can clamp 502 around organ and fluid preservation and transportation system 100 and either its upper container 102B, or its lower container 150A wherein the clamp can have a mechanical system wherein the clamp can open and close around the outer container and can complete a circuit between the drone and the organ and fluid preservation and transportation system. The clamping system can be hinged to stay level during flight in the x and y-axis. The organ and fluid preservation and transportation system can have a parachute attached to it wherein it can be deployed when dropped or the drone can land and deliver the organ and fluid preservation and transportation system safely to the user. The drone or organ and fluid preservation and transportation system can use GPS or other tracking methods to track the container through GPS location, alerts user when device has been dropped off, and/or picked up. In other embodiments, an air powered cooling using vortex directing the air to the heating and cooling system pulling the heat from the system and using the cold air to help cool the system during flight. The organ and fluid preservation and transportation system can be initially cooled before flight by CO2.

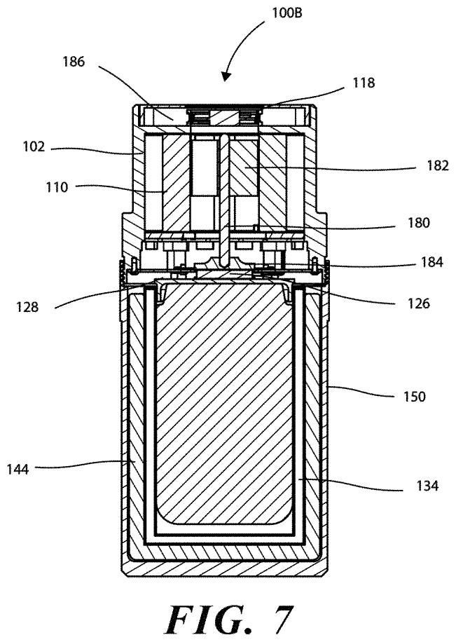

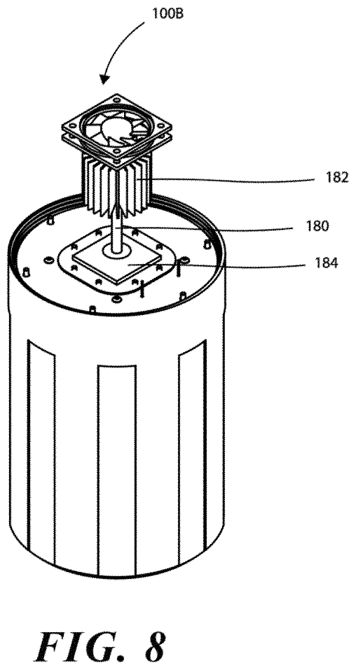

Referring to FIG. 7-8 another embodiment for an organ and fluid preservation and transportation system shown generally at 100B. The organ and fluid preservation and transportation system can have the same system as shown in FIG. 1, but comprise a heat pipe 180, a heat pipe adapter 184, heat sink 182, a fan 118. The heat pipe 180 can be made of copper, stainless steel, aluminum, brass, or other heat sink material wherein the heat pipe can draw the heat away from the heating/cooling module 126. The heat pipe 180 can comprise of tubing or solid material wherein the tubing can have a gas, or a working fluid to move the heat away from the heating/cooling module 126. There can be at least one heat pipe 180. The heat pipe adapter 184 can be connected to a heat pipe and the heating/cooling module 126 wherein the heat pipe adapter can distribute the heat to the heat pipe. The heat sink 182 can be attached to the heat pipe or can touch the heat pipe or can be connected to the heat pipe. The heat sink 182 can be circular, hexagonal, square, triangular or the like in shape with fins that disburse the heat from the heat pipe wherein an optional fan 118 can be connected to or contact with the heat sink 182, and/or heat pipe 180. The fan 118 can be encapsulated by a fan cover 186 wherein the fan cover can have a plurality of holes on its top surface allowing the heat to disburse to the environment.

In other embodiments, the upper container 102 can be replaceable to fit different scenarios for each type of use or storage requirements such as, for example, blood has to be stored from 4 degrees Celsius to 10 degrees Celsius, whereas plasma is usually below −30 degrees Celsius wherein the upper container and the heating/cooling module and the components to cool the system can be changed out to fit the different storage requirements. In addition, with different power requirements for each storage requirement the upper container, lower container, and inner container can vary in size or shape wherein the size the diameter and circumference of each can be at least 1 inch to at least 36 inches.

In embodiments, a method of tracking an organ and fluid preservation and transportation system, blood and its environmental conditions comprising sending a signal to a wireless or wired communicator within the system wherein at least one sensor can send the environmental condition via communicator to the user wherein the sensors can be temperature, humidity, location, pressure, or the like. The communicator can be military bandwidth, or long-range communication, or radio, or the like that can transfer data and information to the user. Tracking the organ and fluid preservation and transportation system through a global positioning system and sending the location to a user. Controlling either remotely, or on the system the environmental condition within the inner container. Alerting user through Bluetooth low energy or through long range communications the user when the upper container is open.

Method of tracking an organ or blood and its environmental conditions from hospital to hospital comprising of a cargo which can be blood, organs, plasma, vaccines, or the like wherein the cargo is scanned with an onboard scanner, once the cargo is scanned the organ and fluid preservation and transportation system time stamps and starts to log the environmental conditions of the container, and the organ and blood wherein the environmental conditions can be sent to a user mobile computing device, or hardwired computing device wherein the user can in real-time track the environmental conditions both inside of the container and outside of the container of the organ and fluid preservation and transportation system. Location of the organ and fluid preservation and transportation system is logged and/or transmitted to the user wherein the user can track via an application on a computing device where the organ and fluid preservation and transportation system is in transit from the initial location to its destination. Alerting the user if the environmental conditions exceed the recommended conditions for the cargo, if the environmental conditions are in its nominal operating conditions for the cargo the user can in real-time see the status either on a mobile computing device or the organ and fluid preservation and transportation system itself through a screen on the system. Once the cargo arrives at its destination the cargo can be scanned again, and the user can see the history of the environmental conditions making sure the cargo stayed within the nominal settings for the cargo allowing the cargo to stay optimally fresh/potent during transportation and tracking its freshness.

Method of tracking biomaterials and alerting a user when opened. Using a System as a Service platform this can track the cargo in the container from when it is removed from the fridge to the container back to the Friday or when it is transfused into someone's body.

FIG. 10 shows another embodiment of a container wherein the container has an outer shell, an insulative layer, a cooling layer, a heat/cool thermal spreading layer, and a second insulative layer to protect the cargo. In addition, the container has a thermal spreading lid that distributes the cooling or heat to the cooling layer and/or heat/cool heat/cool thermal spreading layer. In certain embodiments heat pipes or other means of transferring cold or heat to a system can be wrapped around or can be placed into through channels or next to the cooling layer. The cooling layer can be ice, phase change material, or liquid cooling vessels that can quickly cool the container to the user's set conditions.

Referring to FIG. 10 through 11 another embodiment of an organ and fluid preservation and transportation system shown generally at 10. An organ and fluid preservation and transportation system can comprise a container system 30 and a docking system 12 wherein the container system can comprise an outer body 30, an inner canister 42, and an outer cannister 31 wherein the outer canister can be attached to the inner cannister and the outer canister and the inner canister can be placed into the outer body or attached to the outer body or removably attached to the outer body. Container system 30 can further comprise one or more heat pipe 44 wherein the heat pipes can be attached to a cold bridge 42 wherein the cold bridge can transfer the heat or cold from a heating/cooling module 24 to the heat pipe wherein the heat pipe can be such as, for example standard heat pipes, annular heat pipes, variable conductance heat pipe or the like. The heat pipes 44 can transfer either heat or cold to the outer canister 31, inner canister 42. In certain embodiments phase change material can be placed in the outer body 30 between the outer body and the outer canister and inner canister. The heat pipes can be manufactured from copper, or other efficient material for transferring heat or cool. The container system 30 can further comprise a lid 33 wherein the lid can have one or more holes to allow one or more heat pipes 18 that are connected to the docking system 12 such as the heating/cooling module interface plate 24. The lid 33 can have plugs (not shown) that can plug the one or more holes in the lid.

The docking system 12 can comprise a frame 26 having a base 28 wherein the frame can be any suitable structure that can support the cooling/heating system 11. The cooling/heating system 12 can comprise a heating/cooling module 24 wherein the heating/cooling module is similar to the heating/module as defined above. The docking system 12 can be attached to a vehicle wherein the vehicle can be such as, for example, a humvee, car, helicopter, airplane, delivery truck or the like wherein the docking system can be slaved off of the vehicle's power wherein the vehicle's power can power the cooling/heating system 11. The docking system 12 can further comprise electronics having the same characteristics as defined above. The electronics can regulate the temperature of the container system 30 when the container system is attached to the docking system 12. The docking system 12 can stay stationary while the container system 30 can be removed and stay cold or hot for up to 10 days.

The container system 30 on docking system 12 can be attached to a vehicle, an example is shown in FIG. 6, such as a hum-vee, helicopter, airplane, drone, or other transportation vehicle. docking system 12 can lock the container in place through latch, pressure and other means. While docked it can transfer data and cool the container system 30 and keep it cooled. Locks container onto docking system 12 by handle, lever, clamp, or the like. Can be loaded from the top, bottom, or side. Has computing system and sensors that determines the container system's 30 temperature (environmental conditions), the Phase Change materials current condition, the estimated time to cool back to recommended storage conditions for payload. The cooling/heating system 11 can comprise peltier device, liquid cooling, phase-change cooling, heat pipes and vapor chamber. Depending on type of cooling system it may need heat sinks and fans to dissipate heat away from Peltier device. Runs off batteries, or slaves off of vehicles power. Connection port for electronics and cooling system so that the container assembly can transfer data, track the environmental and container's conditions while the device is docked. Outer Shell (used for protection, grip, insulation). Insulative Layer if necessary, between the outer shell, outer container, inner cooling device, i.e., phase change material, ice packs. Heat pipes or similar to carry the cool or heat from the docking station to the phase change material.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosed embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the embodiments otherwise claimed. No language in the present specification should be construed as indicating any non-claimed element essential to the practice of the disclosed embodiments.

What is claimed is:

1. An organ and fluid preservation and transportation system comprising:
a docking system;
an electrical system; and
and a container system.

2. An organ and fluid preservation and transportation system of claim 1, wherein the docking system comprises a frame wherein the frame supports a heating/cooling system wherein the heating/cooling system can comprise a heating/cooling module wherein a heatsink is attached to the heating/cooling module and a fan attached to the heatsink, one or more heat pipes can be attached to the heating/cooling module.

3. An organ and fluid preservation and transportation system of claim 2, wherein the heating/cooling module is a Peltier device.

4. An organ and fluid preservation and transportation system of claim 1, wherein the container system comprises an outer body wherein an inner container and outer container with an insulative gap between the containers is placed within the outer body and one or more heat pipes are touching the outer container and a phase change material wherein the phase change material is place around the outer container within the outer body.

5. An organ and fluid preservation and transportation system of claim 1, wherein the inner container and outer container are sealed together to create an insulative gap between the layers wherein the insulative gap is evacuated of all its air or filled with argon or similar highly insulative gas.

6. An organ and fluid preservation and transportation system of claim 2, wherein the heat pipes are copper pipes.

7. An organ and fluid preservation and transportation system of claim 1, wherein the electrical system comprises a wireless communication device, a micro-controller, one or more temperature sensors, wherein the electrical system is attached to the docking station or the container system.

8. An organ and fluid preservation and transportation system of claim 7, wherein electronical system can further comprise one or more pressure sensor, light sensor, temperature sensor, accelerometer, scanner.

9. An organ and fluid preservation and transportation system of claim 1, wherein container system can further comprise one or more pressure sensor, light sensor, temperature sensor, accelerometer, scanner.

10. An organ and fluid preservation system of claim 4, wherein a lid is removably attached to the out body wherein the lid has a UV light that when on sterilizes the inside of the container system.

11. An organ and fluid preservation system of claim 7, wherein the electrical system is powered by a solar power array, a vehicle, a power grid or a generator.

12. An organ and fluid preservation system of claim 4 where the inner container and outer container is stainless steel.

13. An organ and fluid preservation system of claim 4, wherein the inner container has a removeable sleeve.

14. An organ and fluid preservation and transportation system comprising:
a docking system;
an electrical system; and
and a container system.
And a means to communicate from the container to a receiver.

15. An organ and fluid preservation and transportation system comprising:
a docking system;
an electrical system; and
and a container system.
And a means to communicate from the docking station or container to a receiver,
And a means to relay position and state of contents to a receiver.
